(19) United States
(12) Patent Application Publication
(10) Pub. No.: US 2020/0178889 A1
(43) Pub. Date: Jun. 11, 2020

(54) SYSTEM AND METHOD FOR DETECTING LEVELS OF PAIN USING MAGNETIC RESONANCE SPECTROSCOPY (MRS)

(71) Applicant: Translational Research Institute Pty Ltd as trustee for Translational Research Insitute Trust, Woolloomgabba (AU)

(72) Inventor: Carolyn Mountford, Robina (AU)

(73) Assignee: Translational Research Institute Pty Ltd as trustee for Translational Research Institute Trust, Woolloongabba (AU)

(21) Appl. No.: 16/707,557

(22) Filed: Dec. 9, 2019

Related U.S. Application Data
(60) Provisional application No. 62/777,560, filed on Dec. 10, 2018.

(51) Int. Cl. A61B 5/00; A61B 5/055; G01R 33/46

(52) U.S. Cl. CPC A61B 5/4824; A61B 5/055; G01R 33/4625; A61B 5/7246; A61B 5/7264; A61B 5/0042

(57) ABSTRACT

One dimensional (1D) and two dimensional (2D) MR spectroscopy of the brain provides an objective test for pain and level of pain. There are two ways of evaluating the data. The first is conventional analysis of the 2D MRS. The second us the use of data mining methods such as testing for correlation between wavelet-based features and self-reported pain intensity, the MBDA method. Both found multiple spectral regions that were highly correlated with self-reported pain. Two of these spectral regions are consistent with changes to the recently assigned substrate α-L Fucose and the Fuc-α(1-2) glycans in the human brain. There are common features recorded compared with prior reports using the MBDA method to evaluate pain associated with spinal cord injury and low back pain. Accordingly, by detecting the levels of selected neurochemical markers in MR spectroscopy of an individual, one can determine the level of pain by comparing spectral data obtained with a reference database which correlates level of selected neurochemicals with levels of pain.

### CROSS-REFERENCE TO RELATED APPLICATIONS

This application claims priority to U.S. Ser. No. 62/777,560, filed Dec. 10, 2018, incorporated by reference herein.

### TECHNICAL FIELD OF THE INVENTION

The present invention relates to a system and method for detecting the level of magnitude of pain being experienced by a subject using magnetic resonance spectroscopy (MRS).

### BACKGROUND OF THE INVENTION

Throughout this application are reference citations to publications. These publications are incorporated by reference herein.

Acute pain is defined by symptoms lasting less than 12 weeks, and chronic pain is defined by symptoms lasting for over 12 weeks.

The McGill Pain Questionnaire, as one of many, can be used to evaluate a person experiencing significant pain. It can be used to monitor the pain over time and to determine the effectiveness of any intervention. It is, however, a subjective means of evaluation.

MRS has been used to detect whether a person is experiencing pain in an objective manner, thereby improving on current methods where each individual completes a lengthy questionnaire to establish a self-reported level of pain.

Seven fucosylated glycans have been assigned in the human brain [1] [2]. These fucosylated glycan molecules have been reported by others to be at end of each neuron. The role of fucosylated glycans in the rat brain has been identified by studies using neuronal cell cultures and immunocytochemistry[3, 4]. These studies reported that the level of fucosylation is highest at the synapse region of the neurons communication[3]. Moreover some of these fucosylated glycans have been shown to be affected by PTSD [5]. Thus, when affected by conditions such as PTSD or pain, they can also affect memory and learning.

For clinicians managing pain it would be useful to provide a new capability if the patients could be monitored on an individual basis to determine response to different types of treatment. It would also be helpful if the level of pain could be gauged in those in the acute phase of pain prior to it transitioning to a chronic pain state, because it becomes more difficult to treat chronic pain than pain in the acute phase.

### SUMMARY OF THE INVENTION

In accordance with the present invention, a system and method for detecting the level, amount, or intensity of pain being experienced by an individual is provided to detect pain in an objective manner, using MRS.

We have now experimental evidence that one or more of these fucose glycans is increased proportionality to self-reported pain whilst others are decreased as consequence of pain. There were two methods of evaluation. The first was by conventional means as described in [5]. These findings were supported by evaluation these specific frequencies being in the region chosen by an MRS biomarker discovery algorithm (MBDA) method as described in [6]. Thus, for the first time it is possible to measure the effect of pain on the human brain in relation to the person's self-reported level of pain.

The invention provides a means of identifying the neurochemical differences between people with a healthy brain experiencing no pain and those with chronic pain or acute pain who have undertaken the subjective evaluation of a pain questionnaire. The resultant 1D and 2D MR spectra can be evaluated by conventional means to measure differences between spectra or by the data mining method known as the MBDA system and method [6] or a like method of data mining such as Statistical Classification Strategy (SCS)[7].

The SCS or MBDA provides an automated ensemble of algorithms for processing raw 1D MR spectroscopy data. The system and method identifies biomarkers using statistical classification algorithms with a high rate of diagnostic accuracy. It may also consist of key modules: post-acquisitional processing, wavelet-based feature extraction (or Fourier Transform), significance testing and feature selection, and statistical classification and cross-validation (FIG. 2). These methods have been used to distinguish control subjects from subjects with spinal cord injury (SCI) as well as subdividing the SCI group into those with and without chronic pain[8]. A Fourier transformed feature extraction and classification algorithm identifies spectral changes was also reported to distinguish control subjects from subjects with chronic low back pain[8].

The invention provides an objective test for chronic pain and acute pain based on neurochemical biomarkers analyzed either by the SCS, MBDA method or other established methods of spectral analysis. It determines if the neuro MRS results are reflective of the presence or absence of pain and the objective level of pain in each individual when compared to the subjective self-reported level of pain.

### BRIEF DESCRIPTION OF DRAWING

FIG. 1 is a plot of the expanded region of a 2D L-COSY spectrum (F2: 4-4.5 ppm, F1: 0.95-1.6 ppm) denoting the assignments of Fuc I to Fuc VII and two α-L-Fucose and lactate. The frequencies identified as reporting on the level of pain are denoted in orange. This is aligned with the regions identified by the MBDA as significant CPPS markers (p<0.01) and markers correlated with CPPS pain score to show pain vs. no pain and level of pain. The frequencies identified as the level of self-reported pain are denoted as shown in FIG. 4.

FIG. 2 is a depiction of an MRS system wherein a 1D MRS Biomarker Discovery Algorithm (MBDA) is used to process MRS data and identify bio markers with diagnostic ability;

FIG. 3 shows scatter plots of classifier features selected by the MBDA. The Sequential Forward Selection technique selected a 3-dimensional feature vector from the entire set of features residing between 0-4 ppm that maximized a statistical measure of class separability between male CP/CPPS patients and controls. The selected features were at 1.19, 1.45, and 2.69 ppm chemical shift. The features at 1.19 and 1.45 ppm were individually significant with correspondingly large effect sizes (1.19 ppm: p=0.0012, IESI=2.01; 1.45 ppm: p=0.0062, IESI=1.42) but the feature at 2.69 ppm, by itself, was not significant (p=0.75, IESI=0.09), indicating that despite its small effect size, the feature at 2.69 ppm provides information orthogonal to the first two features.

FIG. 4 (top) is a plot of the level of pain vs. the intensity at the spectral region at 1.09 ppm; at the spectral frequency of α-L-Fucose and Fuc II[9]; which is a positive correlation (of 0.82); i.e., the level of α-L-Fucose increased with the level of pain. Top is the spectral frequency intensity at 1.09 ppm plotted against the self-reported level of pain with a positive correlation of 0.82. This is the spectral region that includes the substrate α-L-Fucose I Fuc II[9].

FIG. 4 (bottom) is a plot of the level of pain vs. the intensity at the spectral region at 1.42 ppm, of fucosylated glycans Fuc I to Fuc V, which is a negative correlation (of 0.77); i.e., the level of fucosylated glycans Fuc I to Fuc V decreased as the level of pain increased; Bottom is the self-reported level of pain is plotted against the intensity of the resonances at 1.42 ppm where a strong negative correlation of 0.77 is observed. The frequency of 1.42 ppm would include contributions from the fucosylated glycans Fuc I to Fuc V[9].

FIG. 5 shows a system which can be used to practice the invention.

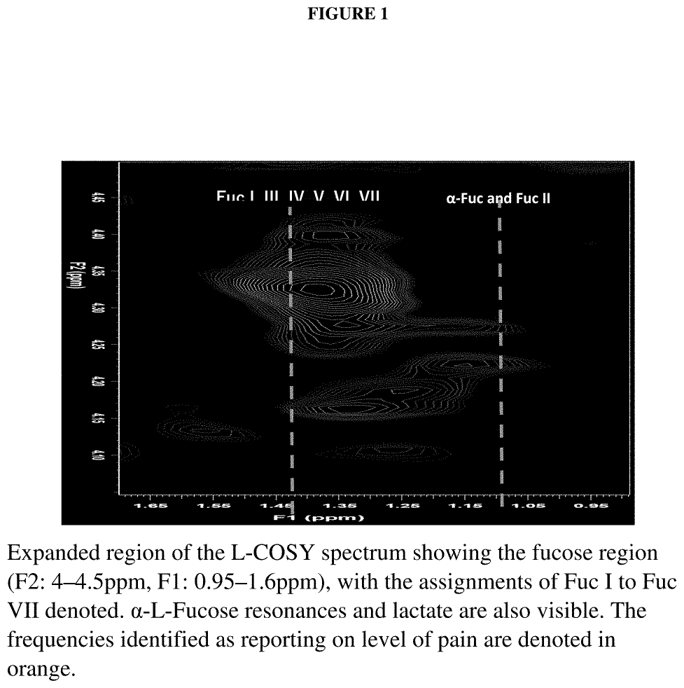

### DESCRIPTION OF THE PREFERRED EMBODIMENT

The invention provides a method for enabling detecting the level of pain experienced by an individual, comprising: obtaining spectral data of the brain of an individual; and comparing the spectral data obtained with reference data which correlates the level of at least one selected neurochemical marker with the level of pain and providing an objective measure to enable detection of the level of pain based on the comparison.

The selected neurochemical marker may be at least one of the substrate α-L-Fucose I and fucosylated glycan denoted Fuc II, the higher level of which indicates a higher level of pain. The selected neurochemical marker may be at least one of the fucosylated glycan denoted Fuc I to Fuc VII, the lower levels of which indicate a higher level of pain. The pain may be of neuropathic or nociceptive or inflammatory in nature from at least one of chronic or acute from low back pain, pelvic pain, pain as a consequence of spinal injury, trauma injury, jaw or dental injury and sporting injury.

The invention provides a system for enabling detecting the level of pain experienced by an individual, comprising: a magnetic resonance spectrometer for obtaining spectral data of the brain of an individual; and a comparator for comparing the spectral data obtained with reference data which correlates the level of at least one selected neurochemical marker with the level of pain and providing an objective measure to enable detection of the level of pain based on the comparison.

The invention provides a system for detecting the response to therapy as deduced by the level of pain experienced by an individual, comprising: a magnetic resonance spectrometer for obtaining spectral data of the brain of an individual; and a comparator for comparing the spectral data obtained with reference data which correlates the level of at least one selected neurochemical marker with the level of pain and providing an objective measure to enable detection of the level of pain based on the comparison.

The selected neurochemical marker may be at least one of the substrate α-L-Fucose I and fucosylated glycan denoted Fuc II, the higher levels of which indicate a higher level of pain. The selected neurochemical marker may be at least one of fucosylated glycan denoted Fuc I to Fuc VII, the lower level of which indicates a higher level of pain. The pain may be at least one of chronic or acute from low back pain, pelvic pain, pain as a consequence of spinal injury, trauma injury, jaw or dental injury and sporting injury.

A preferred embodiment will be described, but the invention is not limited to this embodiment.

There are multiple clinical studies underway.

Magnetic Resonance

Imaging and Spectroscopy: Following screening for MR contraindications, all subjects were examined using a 3T MR scanner (Siemens TIM Trio, or Verio or Prisma or Vida) and a 12 or 32 or 64 channel head coil. The exam consisted of a localizer MRI using 3D MPRAGE which was reconstructed in all three planes for localization of the spectroscopy voxel. Single voxel 1D magnetic resonance spectroscopy (MRS) was acquired from the Posterior Cingulate Gyrus (PCG) using a 3×3×3 voxel and the following parameters were employed: Point-resolved spectroscopy (PRESS) was used with an echo time of 30 ms, repetition time of 2000 ms, 128 averages, and water suppression. A water reference was acquired in the same location using 16 averages and no water suppression. The field homogeneity was optimized for the selected spectroscopy volume of interest by manual shimming to a linewidth of less than 15 Hz for the linewidth at half-height of the unsuppressed water.

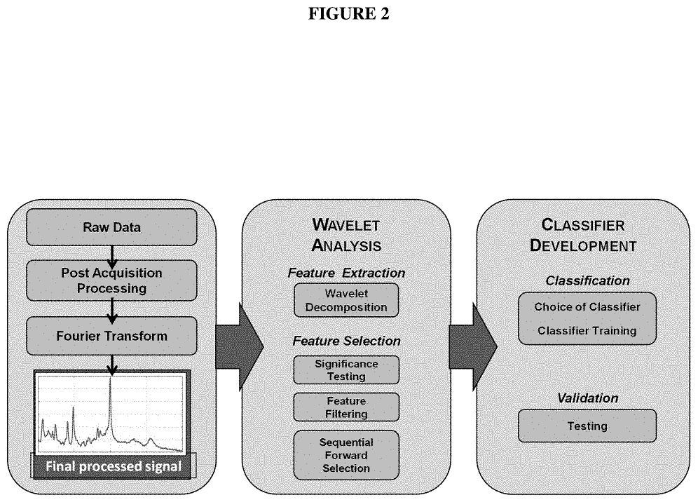

Conventional evaluation of the spectra as described in Quadrelli [5] and Tosh [2]. Wavelet-Based Analysis using MBDA as described in Stanwell [10] and see MBDA (FIG. 2). This is an automated, fully parameterized ensemble of signal processing and machine learning algorithms that consists of four key modules: post-acquisition processing, feature extraction, feature selection and significance testing, and statistical classification and cross-validation.

Results

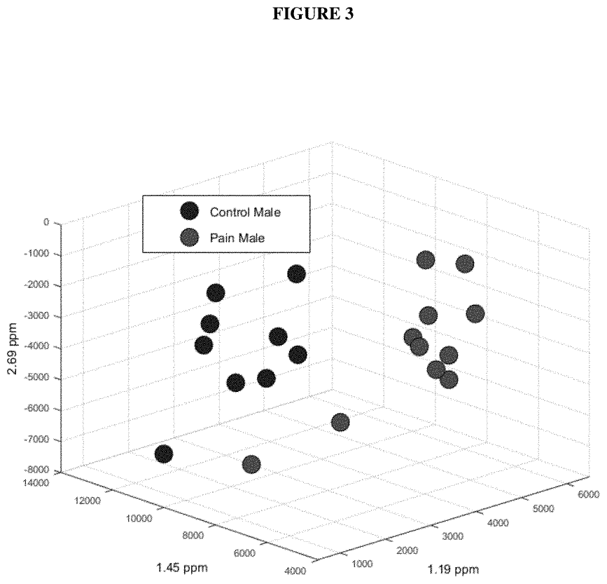

The healthy controls were compared to those with pain and the differences analyzed. Using both conventional spectral analysis, α-L-Fucose I and Fuc II increased with level of pain and Fuc-α(1-2) glycans denoted Fuc I-Fuc VII decreased as the level of pain increased (FIG. 3).

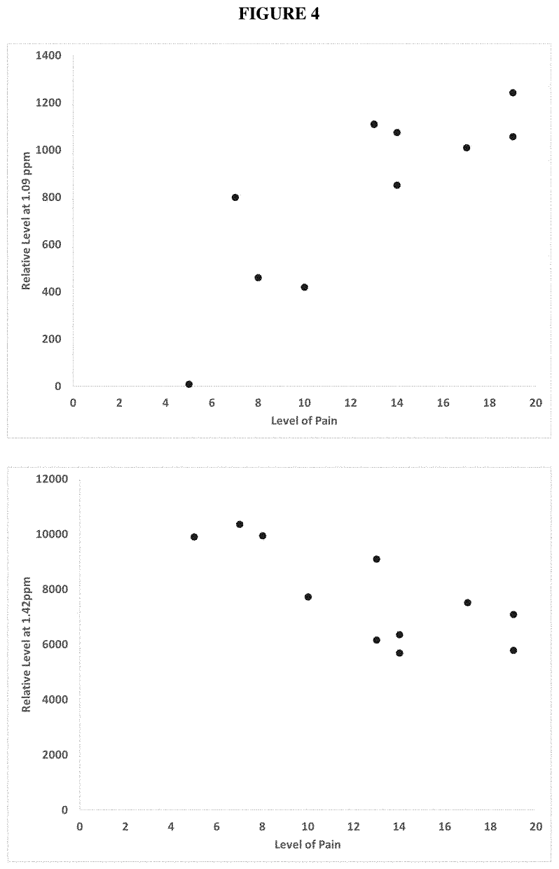

The MBDA method (FIG. 2) selected features were at 1.19, 1.45, and 2.69 ppm chemical shift. Moreover, the features at 1.19 and 1.45 ppm were wavelet-based features at the finest scale, having widths of 0.0088 ppm, whereas the feature at 2.69 ppm was from the coarsest scale having a width of 0.054 ppm. To avoid overfitting with limited training data, higher-dimensional feature vectors were not considered. The three-dimensional feature vector was submitted to Linear Discriminant Analysis (LDA) and Support Vector Machine (SVM) classifiers. Their cross-validated performance in Table 1 documents high sensitivity and specificity. The 3-dimensional scatter plot in FIG. 3 illustrates the ability of the algorithm to separate and discriminate with high degree the pain patients from controls. FIG. 4 shows the plot of the chosen frequencies by both MBDA and conventional spectral analysis versus self-reported pain level.

Since the MBDA method selects the features that are the major discriminators it would appear that pain versus no pain has common features for each of these conditions. The spectral frequencies that are in common for pain versus no pain in spinal cord injury, low back pain and CPPS include 1.19 to 1.21 ppm; 2.1 ppm; and 2.3-2.4 ppm; and 2.64 (CPPS and SCI but not LBP).

Because the common spectral features were seen for CP/CPPS, chronic low back pain and spinal cord injury pain for pain detection, one can fairly conclude that the level of pain (based on amplitude of the neurochemical values) can be detected for chronic low back pain and spinal cord injury pain, as well as trigeminal neuralgia, wherein some data has been obtained to support this.

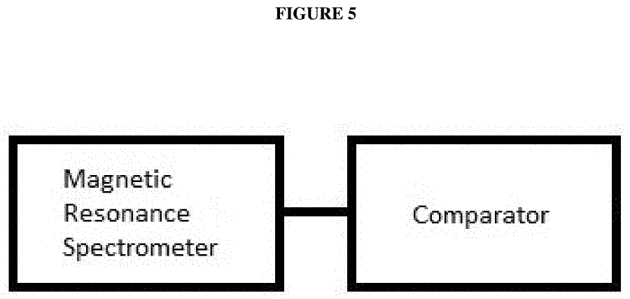

FIG. 5 shows a system which can be used to practice the invention. The system includes a magnetic resonance spectrometer, which could be a Siemens MR scanner as identified above, or any other make and made scanner. The comparator can be an applications program residing on a computer (local or cloud based) which contains a classifier to perform as described herein.

Accordingly, following the method and system described above, one can distinguish between no pain and pain, and also the pain level. The regions will be the frequencies correlating with certain fucosylated molecules and their free substrate.

Although a preferred embodiment has been described, the invention is not limited to this embodiment, and the scope of the invention is defined only by the following claims.

### REFERENCES CITED, INCORPORATED BY REFERENCE

1. Mountford, C., et al., Six fucose-alpha(1-2) sugars and alpha-fucose assigned in the human brain using in vivo two-dimensional MRS. NMR Biomed, 2015. 28(3): p. 291-6.

2. Tosh, N. Q. S., Galloway, G Mountford, C., Two New Fucose-α (1-2)-Glycans Assigned In The Healthy Human Brain Taking The Number To Seven. Nature Scientific Reports In Press.

3. Hsieh-Wilson, L., The Tangled Web: Unraveling the Molecular Basis for Communications in the Brain. Engineering and Science, 2001. 64(2): p. 14-23.

4. Murrey, H. E., et al., Protein fucosylation regulates synapsin Ia/Ib expression and neuronal morphology in primary hippocampal neurons. Proceedings of the National Academy of Sciences of the United States of America, 2006. 103(1): p. 21-26.

5. Quadrelli, S., et al., Post-traumatic stress disorder affects fucose-alpha(1-2)-glycans in the human brain: preliminary findings of neuro deregulation using in vivo two-dimensional neuro MR spectroscopy. Transl Psychiatry, 2019. 9(1): p. 27.

6. Stanwell, P., et al., Neuro magnetic resonance spectroscopy using wavelet decomposition and statistical testing identifies biochemical changes in people with spinal cord injury and pain. NeuroImage, 2010. 53(2): p. 544-52.

7. Lean, C. L., et al., Accurate diagnosis and prognosis of human cancers by proton MRS and a three-stage classification strategy, in Annual Reports on NMR Spectroscopy. 2002, Academic Press. p. 71-111.

8. Siddall, P. J., et al., Magnetic resonance spectroscopy detects biochemical changes in the brain associated with chronic low back pain: a preliminary report. Anesth Analg, 2006. 102(4): p. 1164-8.

1. A method for enabling detecting the level of pain experienced by an individual, comprising:
obtaining spectral data of the brain of an individual; and
comparing the spectral data obtained with reference data which correlates the level of at least one selected neurochemical marker with the level of pain and providing an objective measure to enable detection of the level of pain based on the comparison.

2. The method of claim 1, where in the selected neurochemical marker is at least one of the substrate α-L-Fucose and fucosylated glycan denoted Fuc II, the higher level of which indicates a higher level of pain.

3. The method of claim 1, wherein the selected neurochemical marker is at least one of the fucosylated glycan denoted Fuc I to Fuc VII, the lower levels of which indicate a higher level of pain.

4. The method of claim 1, wherein the pain is of neuropathic or nociceptive or inflammatory in nature from at least one of chronic or acute from low back pain, pelvic pain, pain as a consequence of spinal injury, trauma injury, jaw or dental injury and sporting injury.

5. A system for enabling detecting the level of pain experienced by an individual, comprising:
a magnetic resonance spectrometer for obtaining spectral data of the brain of an individual; and
a comparator for comparing the spectral data obtained with reference data which correlates the level of at least one selected neurochemical marker with the level of pain and providing an objective measure to enable detection of the level of pain based on the comparison.

6. A system for detecting the response to therapy as deduced by the level of pain experienced by an individual, comprising:
a magnetic resonance spectrometer for obtaining spectral data of the brain of an individual; and
a comparator for comparing the spectral data obtained with reference data which correlates the level of at least one selected neurochemical marker with the level of pain and providing an objective measure to enable detection of the level of pain based on the comparison.

7. The system of claim 5, wherein the selected neurochemical marker is at least one of the substrate α-L-Fucose I and fucosylated glycan denoted Fuc II, the higher levels of which indicate a higher level of pain.

8. The system of claim 5, wherein the selected neurochemical marker is at least one of fucosylated glycan denoted Fuc I to Fuc VII, the lower level of which indicates a higher level of pain.

9. The system of claim 5, wherein the pain is at least one of chronic or acute from low back pain, pelvic pain, pain as a consequence of spinal injury, trauma injury, jaw or dental injury and sporting injury.
